(19) United States
(12) Patent Application Publication
(10) Pub. No.: US 2009/0104160 A1
(43) Pub. Date: Apr. 23, 2009

(54) Mobilization of Stem Cells After Trauma and Methods Therefor

(75) Inventors: Henry E. Young, Macon, GA (US); Asa Black, Macon, CA (US)

Correspondence Address: FISH & ASSOCIATES, PC;ROBERT D. FISH, 2603 Main Street, Suite 1000, Irvine, CA, 92614-6232, US

(73) Assignee: MORAGA BIOTECHNOLOGY CORPORATION, Los Angeles, CA (US)

(21) Appl. No.: 12/024,841

(22) Filed: Feb. 1, 2008

Related U.S. Application Data
(60) Provisional application No. 60/899,261, filed on Feb. 1, 2007.

(51) Int. Cl. C12Q 1/06; A61K 35/12

(52) U.S. Cl. 424/93.7; 435/39

(57) ABSTRACT

Methods are presented in which release of stem cells from skeletal muscle is quantitated and correlated with severity of a disease or trauma, a future treatment option, prognosis, and/or anticipated time to recovery. Most preferably, the stem cell is a BLSC and/or an ELSC, and the stem cell isolation for the cell count is performed using sedimentation or filtration as principal separation step, thereby avoiding commonly used complicated, expensive, and time-consuming processes such as antibody-based separation and fluorescence-activated cell sorting.

This application claims priority to our copending U.S. provisional application with the Ser. No. 60/899,261, which was filed Feb. 1, 2007 and is incorporated herewith in its entirety.

### FIELD OF THE INVENTION

The field of the invention is stem cells, and especially adult blastomere-like stem cells (BLSC) and their use in diagnosis and prognosis of disease or trauma.

### BACKGROUND

Over the last decades, numerous types of stem cells have been discovered, and depending on the particular source and/or inducibility, various uses have been proposed. In most instances, stem cells are proposed to help regenerate diseased or otherwise dysfunctional tissue or organs. Thus, the focus of current research is in the generation of stem cells and their introduction into a human in an attempt to mitigate or treat a disease.

Recently, it was reported that certain developmentally limited, neural tissue-committed stem cells (TCSCs) are mobilized from murine bone marrow into peripheral blood following stroke, and that the TCSCs are then chemoattracted to the damaged neural tissue in an SDF-1-CXCR4, HGF-c-Met-, and LIF-LIF-R-dependent manner (Leukemia (2006) 20, 18-28). Similarly, Steele et al published that cardiac progenitor-like cells were released from cardiac tissue, had the potential for re-implantation and would be capable of undergoing injury-induced differentiation (J Heart Lung Transplant 2005 November; 24(11): 1930-9). Condon et al. reported that selected surgical procedures induced the mobilization of circulating endothelial progenitor cells, and Conden then addressed potential implications of such cells in residual and metastatic tumor growth during the perioperative period as such cells are thought to incorporate into foci of tumor neovascularization to increase tumor growth (Surgery 2004 June; 135(6):657-61).

Elsewhere, Gill et al. reported (Circ. Res. 2001; 88; 167-174) that vascular trauma may induce release of endothelial precursor cells from bone marrow, which will then lead to the appropriate tissue repair, and Yokote et al. postulated (J Atheroscler Thromb 2003; 10(4):205-210) that bone marrow derived circulating progenitors of intimal smooth muscle cells may be used for neointimal formation after injury. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

While such discoveries provide valuable insights into specific disease related events with regard to progenitor or precursor cells, the general conclusion in the field is that postnatal bone marrow (or certain non-marrow tissue) may harbor a non-hematopoietic population of cells that may account for beneficial effects of tissue regeneration of corresponding injured tissue, thereby again pointing back to yet another potential reservoir of committed stem cells for reconstitutive or curative therapy.

In still further known methods, certain progenitor and precursor cells can be mobilized from the bone marrow using specific chemical stimuli, and especially colony-stimulating factors (e.g., colony-stimulating factor, GM-CSF [granulocyte-macrophage colony-stimulating factor] or G-CSF [granulocyte colony-stimulating factor]), or specific physical and non-destructive stimuli (e.g., bone surgery and/or surgical implantations, ultrasound, ultrasound shockwaves, pulsed electromagnetic field (PEMF) therapy, CAT scan, and magnetic resonance imaging (MRI) as described in U.S. Pat. App. No. 2006/0051328. Once more, all of the above known methods and procedures are used to generate stem cell that are then contemplated for use in a therapeutic treatment.

Therefore, while numerous methods and compositions of stem cells are known in the art, all or almost all of them suffer from one or more disadvantages, and/or fail to address heretofore unmet needs. Consequently, there is still a need to provide stem cell related compositions and methods.

### SUMMARY OF THE INVENTION

The concept that primitive stem cells, and especially pluri- or totipotent stem cells from adult skeletal muscle participate in wound repair and regeneration remains highly controversial and the identities of the particular cell type(s) involved remains largely unknown. Previous studies by the inventors identified two categories of primitive pluripotent stem cells within adult skeletal muscle, blastomere-like stem cells (BLSCs) and epiblast-like stem cells (ELSCs). Their identification was based on cell size, cell surface markers, and differentiation potentials, and the inventors now discovered that these stem cells are mobilized into peripheral blood in substantial quantities as a result of trauma and other injuries and/or disease states, and that such mobilization can be used as a diagnostic and/or prognostic tool in the assessment and treatment of numerous conditions and diseases.

Therefore, in one aspect of the inventive subject matter, a method of medical evaluation includes a step of ascertaining that a patient has a trauma or disorder, and a step of obtaining at least one peripheral and/or skeletal muscle stem cell count in the patient. In a further step, the stem cell count is correlated with the severity of a disease or trauma, a future treatment option, a prognosis, and/or an anticipated time to recovery.

Most preferably, the stem cell count is obtained from peripheral blood, and the stem cells are BLSCs, BE-TRSCs (transitional form between BLSC and ELSC), ELSCs, EG-TRSCs (transitional form between ELSC and GLSC), or GLSCs. In most typically embodiments, the patient is a human, and the disease is a chronic disease (e.g., inflammatory disease, autoimmune disease, chronic bacterial or viral disease, etc.) or an acute trauma (e.g., accident, surgery, etc.). While numerous manners of enriching stem cells for contemplated cells counts are deemed suitable, it is especially preferred that enrichment substantially exclusively relies on sedimentation, filtration, and/or non-optically assisted electrostatic separation.

Particularly contemplated patients are human or non-human mammals, and the disorder or trauma may be treated by administration of contemplated stem cells. Stem cells are preferably obtained (for stem cell count and/or treatment) by obtaining whole blood from a patient and allowing the whole blood to sediment for a time sufficient to separate non-stems cells from the stem cells, and by withdrawing the stem cells from the so formed supernatant of the whole blood. The cells may then be counted in a cell counter and/or used in a fluid carrier as a therapeutic entity (typically after further purification, cryoactivation, and induction). Blood collection is preferably performed by venipuncture and collection in an evacuated tube, typically comprising a complexing agent (e.g., EDTA), and storage of the so collected whole blood is most preferably at a temperature below ambient temperature (most typically between 4° C. and 20° C.). Under most circumstances, so prepared supernatant will surprisingly comprise a nearly pure fraction of stem cells (BLSC, BE-TRSC and ELSC), often at less than 1% content of non-stem cells.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1 is an exemplary graph indicating release of pluripotent stem cells from skeletal muscle into peripheral blood in response to trauma.

FIG. 2 is an exemplary graph indicating increase in the circulating stem cell levels in peripheral blood was observed following mild stress.

### DETAILED DESCRIPTION

The inventors have discovered that release of various stem cells, and especially BLSCs, ELSCs, and to some extent germ layer lineage stem cells (GLSCs), from skeletal muscle may be employed as a diagnostic and/or therapeutic marker for various diseases and conditions as such cells are released into the circulation in a trauma dependent manner. For example, BLSCs cells are mobilized in mammals (e.g., pig) immediately following trauma from the skeletal muscle into the circulatory system in remarkably large numbers (e.g., 22×10̂6/ml pre-trauma to 512×10̂6/ml post trauma as measured in serum).

Among other preferred embodiments considered herein, such finding is expected to find correlation in human, where quantitative and kinetic analysis of stem cell mobilization may be used in staging, diagnosis, and/or prognosis of trauma victims, and/or in patients diagnosed with a variety of diseases (e.g., inflammatory disease, auto-immune disease, etc.). Similarly, it should be appreciated that the findings, considerations, and contemplated uses will also apply to non-human animals, and especially non-human mammals and non-human vertebrates. With respect to BLSCs, ELSCs, and GLSCs, numerous examples, isolation protocols, and culture conditions are provided in our co-pending applications with the serial numbers PCT/US05/30284 (published as WO 2006/028723), and PCT/US07/05142 (published as WO 2007/100845), both of which are incorporated by reference herein.

For example, in one particularly preferred aspect of the inventive subject matter, absolute numbers and release kinetics of release of adult stem cells (and most preferably BLSCs, ELSCs, and/or GLSCs) into the circulatory system are observed and the results are then correlated with the severity of injuries sustained by human trauma patients. For example, blood samples from patients will be obtained in predetermined intervals, with the first blood sample typically drawn upon admission or first presentation, and with one or more samples being drawn after one to several hours, or after one to several days (or even weeks). Most commonly, where the patient is admitted with an acute disorder or trauma, staging, therapeutic strategy, and/or prognosis will be based on the initial stem cell count. On the other hand, where the patient is admitted with a chronic disorder, several stem cell counts can be performed to establish a trend or variability of results.

In especially preferred aspects of the inventive subject matter, the stem cell count will be exclusively based on a BLSC count obtained from blood as such determination can be performed in a remarkably rapid and reliable manner. As BLSCs are characterized by their extremely small size, positive trypan blue staining, and highly refractile appearance under phase contrast, a BLSC count can be readily obtained from plasma fraction using trypan blue staining and a hematocytometer. Alternatively, and especially where the patient is in recovery, circulating transitional cells (from BLSC to ELSC) and/or ELSCs may be included in the cell count. In less preferred aspects, it should be noted that GLSCs may also be employed in the cell count.

Of course, it should be noted that the stem cell count may not only be based on a numeric analysis of a single stem cell type, but may also include a calculation of a numeric ratio of one stem cell type relative to another stem cells type. For example, the stem cell count may include a numeric ratio between BLSCs and ELSCs, or a numeric ratio between BLSCs and transitional cells, and/or a numeric ratio between one or more types of stem cells and non-stem cells. For example, and especially where the disease has an inflammatory component, a ratio between stem cells and selected lymphocytes may be employed. On the other hand, where the disease was a severe trauma, a ratio between stem cells and erythrocytes and/or thrombocytes may be used.

Numerical analysis of stem cells or ratios will typically be based on a base line count that is representative of a healthy patient and will as such have an absolute point of reference, but is should also be appreciated that dynamic changes of stem cell counts may also be suitable. For example, as minor injuries generally trigger only relatively small releases (e.g., 1.5 to 4-fold increase of circulating stem cells in blood) of stem cells into the systemic circulation, and severe trauma will trigger massive releases (e.g., 15 to 20-fold increase of circulating stem cells in blood) of stem cells into the systemic circulation, an increase of circulating stem cells will thus be indicative of deterioration of a patient's condition (e.g., internal bleeding, rupture of internal organ, etc.). On the other hand, a decrease of circulating stem cells may be indicative of healing or plateauing of a change in condition. Similarly, determination of a change in stem cell ratios (stem cell to stem cell and/or stem cell to non-stem cell) is contemplated to provide additional insight into staging, healing progress, effectiveness of drug treatment, and/or prognosis. For example, a decrease in the ratio of circulating stem cells to leukocytes may be indicative of therapeutic effect of a drug treatment while an increase ratio of circulating stem cells to leukocytes may point to worsening of a condition.

Moreover, it should be noted that the stem cell count may also include numerical and/or dynamic assessment of the stem cells in the skeletal muscles (or other tissues). Most typically, circulating stem cell quantities are inversely proportional to resident stem cells (and especially to stem cells in muscle tissue). Therefore, it should be appreciated that a determination of the stem cell count may be done by muscle biopsy and/or by count of the circulating stem cells. With respect to the location of the muscle from which a biopsy is taken, it should be appreciated that numerous locations are deemed suitable and especially preferred muscles include skeletal muscles that allow ready access (e.g., rectus femoris, vastus medialis, rectus abdominis, biceps femoris, etc.). Still further, it should be noted that while skeletal muscles are especially preferred as source material for stem cell counts, non-skeletal muscles and even non-muscle tissues are also deemed suitable. For example, tissue-based stem cell counts may be based on peristaltic muscles, cardiac muscle, etc., while non-muscle tissues especially include adipose tissue.

In a further preferred aspect of the inventive subject mater, it is contemplated that patient assessment can also be performed on the basis of a ratio of circulating stem cells (and most preferably BLSCs) to stem cells obtained from a tissue, and most typically from skeletal muscle. Such analysis may advantageously provide information on the severity of trauma, stem cell reserve capacity, and/or chronic nature of a condition. For example, where ratio of circulating stem cells to muscle cell stem cells is relatively high (e.g., >10-fold), the disorder is often an acute trauma. On the other hand, where the ratio is lower and where the total stem cell count in the respective compartments is relatively low (about 5×10̂7/ml in blood 2×10̂7/g tissue), the condition may be characterized as a chronic disease.

There are numerous manners of counting stem cells known in the art, and exemplary methods of counting stem cells from a tissue sample and from whole blood are provided in the experimental section below. Further methods of stem cell counting are described in our co-pending applications with the serial numbers PCT/US05/30284 (published as WO 2006/028723), and PCT/US07/05142 (published as WO 2007/100845). Therefore, it should be appreciated that particularly preferred methods will substantially exclusively rely on stem cell separation using sedimentation, filtration, and/or electrostatic particle size separation. As provided in more detail below, and where time constraints allow, stem cells may be separated in a collection vial via gravity over several hours or even days. On the other hand, where a more expeditious separation is required, stem cells may be at least partially separated from non-stem cells via centrifugation preparation of a plasma layer. Similarly, due to the very small size of the stem cells, filtration or electrostatic particle separation of a whole blood sample may be suitable. While purities of better than 90%, and more typically better than 95% and even better than 99% may be obtained with such relatively simple methods, it should be noted that quantitative stem cell separation is often not required as the stem cells exhibit several unusual and readily identifiable characteristics (see above).

In less preferred aspects, the stem cell count may also include antibody-based methods, and especially those in which separation is based on magnetic beads or fluorescence-activated cell sorting. It should be noted that such methods are also suitable to determine the relative ratios between stem cells presented herein. For example, antibodies with specificity against CD90 may be used to identify GLSCs, antibodies with specificity against CD10 may be used to identify ELSCs, and antibodies with specificity against CD66e may be used to identify BLSCs in the blood samples. In such case, no prior separation of stem cells from non-stem cell is typically needed.

Regardless of the manner of how the stem cell count was obtained, it is contemplated that the stem cell count(s), ratio(s), kinetic(s), and/or stem cell distribution(s), is/are then correlated with a trauma or disease severity, recovery status and/or progression, and prognosis. Among other contemplated results, it is expected that trauma or disease severity will generally correlate (in a linear or non-linear fashion) with the degree of stem cell mobilization, and most typically with the degree of mobilization from skeletal muscle to the circulation. Therefore, a higher stem cell count in whole blood and lower stem cell count in skeletal muscle will typically be indicative of a more severe condition. On the other hand, a reduction of peripheral stem cell count with concomitant increase in skeletal muscle stem cell count will typically be indicative of recovery from the disease or condition. In further examples, it is contemplated that gradually decreasing skeletal muscle stem cell count and/or increasing peripheral stem cell count will often be associated with a less favorable prognosis (or treatment efficacy). Therefore, it should be appreciated that such results can be used not only to guide triage decisions and treatment options, but also to monitor progress and/or effectiveness of treatment of a patient.

Therefore, in another preferred aspect of the inventive subject matter, a decision to commence, modify, or terminate treatment of a disease or condition is guided at least in part by the number and/or kinetic of stem cell mobilization into peripheral blood. For example, where a chronic disease is associated with an elevated number of circulating BLSCs, and where the elevated number of circulating BLSCs fails to come down after administration of a suitable drug, the drug dose may be increased or the drug type modified.

While it is generally preferred that the stem cells under observation are BLSCs, BE-TRSCs, ELSCs, EG-TRSCs, and/or GLSCs, other progression states are also deemed suitable and include committed precursor cells, blast cells, etc. Similarly, it is preferred that the stem cells are monitored in peripheral blood and that the source of mobilization is from skeletal muscle. However, in alternative aspects of the inventive subject matter, it is generally contemplated that the stem cells may be monitored in any organ or compartment other than that from which they originate. For example, where the stem cells originate in connective or adipose tissue, they may be monitored at the site of injury (e.g., using a biopsy or smear). While monitoring is preferable performed ex vivo using optionally labeled antibodies and suitable detection techniques (e.g., isolation and manual count, or ELICA, FACS, immuno-magnetic assays, etc.) well known in the art, certain in vivo detections are also contemplated and include all radiographic methods known in the art. Particularly preferred in vivo methods include PET and SPECT spectroscopy. Most typically, intervals at which the mobilized stem cell population is measured lies between about 2 hours to 24 hours, however short intervals (e.g., between 5 and 120 minutes) are also contemplated. Alternatively, and especially where monitoring is over a relatively long period (e.g., for two weeks) or longer intervals are also deemed suitable (e.g., biweekly). Of course, it should be recognized that mobilization of the stem cells from an originating tissue to a second tissue (or compartment) may be measured in both tissues. Thus, measurement may be based on a decrease in stem cells in an originating tissue (e.g., skeletal muscle) and/or be based on an increase in stem cells in the second tissue (e.g., blood).

In yet a further preferred aspect of the inventive subject matter, it is contemplated that stem cells of a patient (and especially BLSCs, BE-TRSCs, and ELSCs) could be isolated, optionally stored and/or manipulated to accelerate the healing process of a patient in which recovery or healing is delayed due to low numbers of stem cells. Such approach could lead to dramatic advances in the care and treatment of trauma patients, particularly where autologous or HLA compatible stem cells are already available from a previously isolated population of such cells (e.g., banked, cultured, etc.). Administered numbers of cells may vary considerably, however, will generally be at least 10̂5, more typically at least 10̂6, and most typically at least 10̂7.

### EXPERIMENTS

### Porcine Study

Tissue Harvest: Adult pigs (n=20) were anesthetized with tiletamine and zolazepam, then prepared for surgery with a Betadine wash and draped in a sterile fashion. A 10-ml pre-trauma blood sample was obtained from the internal jugular vein, placed in a standard hemovac tubes containing EDTA. A midline laparotomy incision was performed, a piece of the right rectus abdominis muscle was obtained (pre-trauma), placed into container with ice-cold transport solution (MBC-PGB-MED-100-A004, Moraga Biotech Corp. 1061 Moraga Drive, Los Angeles, Calif. 90049, USA). The spleen was isolated, resected and removed. The abdominal aorta was isolated above the celiac trunk and below the renal arteries for insertion of a vascular cannula for infusion of a pancreatic protective solution, i.e., ice cold Hank's Buffered Salts Solution (GIBCO, Grand Island Biological Company, Grand Island, N.Y.), pH 7.4. The pancreas was then resected.

A piece of left rectus abdominis muscle was obtained (post-trauma) and placed into ice-cold transport solution (MBC-PGB-MED-100-A004, Moraga Biotech Corp.). A 10-milliliter post-trauma blood sample was obtained by venipuncture from the internal jugular vein and placed in a standard hemovac tube containing EDTA. The pigs were euthanized. The time period from pre-trauma blood draw to post-trauma blood draw approximated 90 minutes. Blood was collected under sterile conditions into VACUTAINER™ (Evacuated blood collection tubes, Becton Dickinson, USA) containing EDTA to prevent clotting. The tubes were invert several times to ensure proper mixing and stored in wet ice at 4° C. until processed. Rectus abdominis tissue samples were placed into ice cold transport solution (MBC-PGB-MED-100-A004, Moraga Biotech Corp.) and stored at 4° C. in wet ice until processed. This procedure of splenectomy followed by pancreatectomy represented the trauma model.

Isolation of Adult Stem Cells from Whole Blood: One-half ml of whole blood was added to 49.5 mls of hemolysis solution (MBC-ASB-REBG-900A-001, Moraga Biotech Corp.) in a 50 ml conical tube. The tube was inverted twice to mix. The tubes were balanced and centrifuged at 1800×g for 10 minutes. The supernatant was removed by aspiration. The cell pellets were resuspended by agitation (by stroking across an Eppendorf tube holder). The cell suspension was reconstituted with 2 mls of reconstitution solution (MBC-ASB-REBG-900A-002, Moraga Biotech Corp.). To each ml of reconstituted cells, 49 mls of clarification solution (MBC-ASB-REBG-900A-003, Moraga Biotech Corp.) was added. The tubes were inverted twice to mix. The tubes were balanced centrifuge at 1800×g for 10 minutes. The supernatant was removed by aspiration and the pellets resuspended by agitation. The cell suspension was reconstituted with 2 ml serum-free defined BLSC adherent propagation medium (MBC-PGB-MED-1A00-A006, Moraga Biotech Corp.) and cell counts performed as described below.

Alternative and Simplified Isolation from Whole Blood: Whole blood is withdrawn by venipuncture and placed into a tube containing EDTA (15% solution), which is inverted several times to mix the blood with the EDTA. The tubes are then placed in a refrigerator in a vertical position at 4° C. for a minimum of 48 hours (humans), or a minimum for non-human of about 72-96 hours dependent on the species. The red blood cells, white blood cells, macrophages, GLSCs, and most ELSCs will sediment to the bottom of the tube, leaving the plasma and the BLSCs and BLSC-to-ELSC transitional stem cells (BE-TRSCs) in suspension. Within 48-96 hours the plasma is withdrawn from the tubes to so obtain an almost homogenously purified population of BLSCs (typically less than 4%, more typically less than 2%, and most typically less than 1% non-stem cells) in the withdrawn plasma. The BLSCs can then be stored in plasma for at least 3 months (at 4° C.) while still maintaining viability for cell culture.

Remarkably, the inventors also observed that storage of the BLSCs and BE-TRSCs in the cold (4° C.) will activate the cells, allowing them to attach to a (type-I collagen) substratum and to be induced to differentiate into any number of cell types. So stored cells may also be used as implant material into diseased, aged, or otherwise compromised tissue for tissue regeneration or augmentation.

Isolation of Adult Stem Cells from Solid Tissues: Rectus abdominis tissue samples were processed as follows. 50-ml conical polypropylene tubes were labeled appropriately, 5 ml of transport solution (MBC-PGB-MED-100-A004, Moraga Biotech Corp.) added and the tubes weighed to determine tare weight. Using sterile procedures the tissue samples were removed from their containers and separated into pieces approximating <5 mm³. The pieces of tissue were then added to the 50-ml conical tubes up to the 10 ml line on the tube. The tube was weighed for final weight. Tare weight was subtracted from final weight to determine the weight of the tissue per tube.

Under sterile conditions, about half of the tissue and transport solution were removed from the conical tube and placed into sterile 60-mm glass Petri dish. The tissue was minced into fine pieces the consistency of orange marmalade using small scissors and forceps. The tissue mince was poured into a fresh (labeled) 50-ml conical tube. The procedure was repeated with the second half of the tissue pieces and solution. This resulted in approximately 5 ml of minced tissue per 50 ml conical.

Ten-milliliters of serum-free defined tissue release solution (MBC-PGB-RED-100-A003, Moraga Biotech Corp.) were added to each 50-ml conical tube. The remainder of the tube was filled with transport solution. The caps were tightened and parafilmed to seal the tubes. Each tube was placed within individual zip-closure plastic bags. The bags were rolled around the tubes. The bags were zip-closed and sealed with tape. The tubes in sealed bags were placed within an Orbit Incubator Shaker (Labline) for 18 hours at 37° C. at a speed of 100 rpm. At termination of digestion the bags were removed from the incubator/shaker, the outside of the bags was disinfected (using MBC-ASB-MSD-900-A002, Moraga Biotech Corp.), the tubes were removed from the bags, and the exterior of the tubes was disinfected.

The 50-ml tubes were centrifuged at 25×g for 10 minutes. The tubes were checked for pelleted material. In some instances undigested tissue remained in the tubes. The supernatants were removed and placed into fresh 50-ml conical tubes and centrifuged at 1800×g for 10 minutes. The supernatants were discarded to disinfectant solution (MBC-ASB-MSD-900-A001, Moraga Biotech Corp.). Pellets were resuspended by agitation and reconstituted with 2-ml of serum-free defined BLSC adherent propagation medium (MBC-ASB-REBG-900A-007, Moraga Biotech Corp.). Cell counts were performed as described below.

Cell Counts: Final volume of each cell suspension was determined and recorded. Fifteen microliters of each cell suspension was removed and placed into separate 2.0 ml polypropylene tubes. Fifteen microliters of sterile 0.4% Trypan Blue solution (MBC-ASB-MSD-900-A005, Moraga Biotech Corp.) was added to each tube. The contents were mixed by trituration 5-6 times and 15 μl of cell suspension/trypan blue was placed on a hemocytometer for cell counting. The cells were initially identified by size (typically equal or less than 5 microns for BLSC and BE-TRSCs, and typically less than 2 microns for BLSCS) and their staining pattern with trypan blue. Totipotent stem cells are very small spherical entities, typically less than 2 micron, that are trypan blue positive and stain only for the CEA-CAM epitope. Totipotent transitional stem cells are slightly larger spherical entities typically between 2 and 5 micron that are trypan blue positive along their periphery and trypan blue negative within the center of the spheres. They contain cell surface epitopes for both CEA-CAM and SSEA-4. Pluripotent stem cells are small spherical entities, typically 5-8 micron, that are trypan blue negative and stain for only the SSEA-4 epitope. Pluripotent transitional cells are intermediate in size between the pluripotent stem cells and the germ layer lineage stem cells. They are trypan blue negative and have cell surface epitopes for both SSEA-4 and Thy-1. Germ layer lineage stem cells (GLSCs) are larger irregularly-shaped entities that are trypan blue negative and contain the Thy-1 epitope.

All single completely trypan blue-positive cells within the nine large boxes of a standard hemocytometer were counted and then averaged for the number of totipotent stem cells per each large box. The identities of the cells were later verified using antibodies to CEA-CAM and SSEA-4. The formula to determine final cell number per ml was [(((average number)/5)/5)×0.25)×2]=cells×10⁶ cells per ml. Final cell number calculations were based on number of cells per ml for whole blood or number of cells per gram of tissue for the rectus abdominis tissue samples.

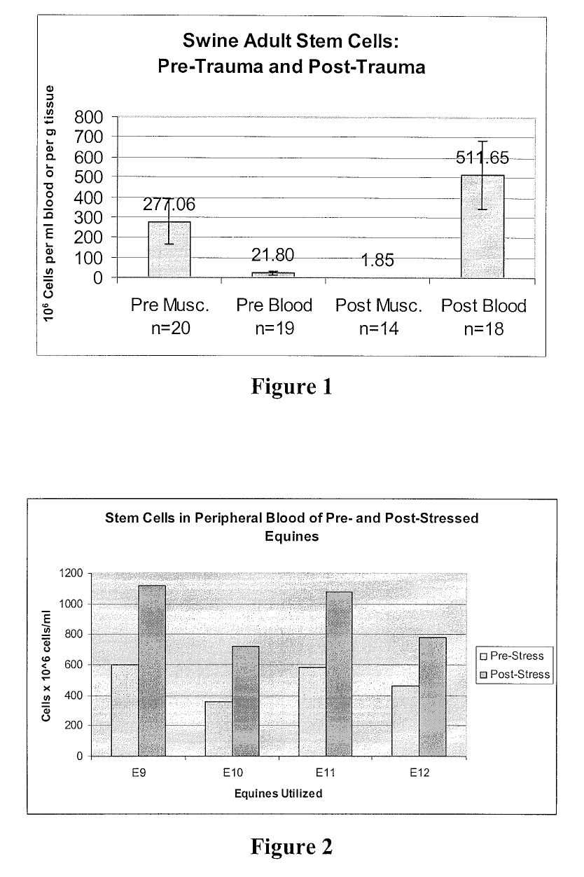

Results: Pre-trauma adult porcine skeletal muscle initially contained 277.1 million adult stem cells per gram of tissue. Following the 90 min surgery, levels decreased to 1.8 million cells per gram of tissue. In contrast, pre-trauma porcine blood initially contained 21.8 million adult stem cells per ml of blood. Following the 90 min surgery, levels increased to 511.6 million cells per ml of blood. These results support the hypothesis that primitive adult pluripotent stem cells residing in adult skeletal muscle are mobilized to the peripheral blood, where they assist in the repair and replacement of damaged tissues. FIG. 1 graphically illustrates the significant changes of cells in the respective compartment pre- and post-trauma.

Equine Study

Equine blood was obtained by venipuncture following standard acceptable veterinary practices. Four ml of blood was withdrawn prior to mild stress and after mild stress using sterile procedures and placed into tubes containing a 15% EDTA solution. The tubes were inverted three-four times to mix the blood with the EDTA and then placed into refrigeration at 4° C. for 48 hours.

After 48 hours of gravity separation, the blood had separated into a floating plasma fraction and a sedimented cellular fraction. The cellular fraction contained red blood cells, white blood cells, most ELSCs, GLSCs, and hematopoietic stem cells. The plasma fraction was withdrawn by a sterile pipette to a second sterile tube and stored under refrigeration at 4° C.

Fifteen microliters of the plasma fraction from each equine was mixed with 15 microliters of sterile 0.4% Trypan blue, the resultant solution mixed, placed onto a hemocytometer and the isolated cells counted and photographed. Blastomere-like stem cells (BLSCs) are Trypan blue positive and <2.0 microns in size. Blastomere to epiblast transitional cells (BE-TrSCs) are both Trypan blue positive & negative and 3-5 microns in size. Epiblast-like stem cells (ELSCs) are Trypan blue negative and 6-8 microns in size.

As can be taken from FIG. 2, a statistically significant increase in the circulating stem cell levels in peripheral blood was observed following mild stress (10 minutes of cantering). Thus, mild stress led to an about doubling of circulating stem cells, which is a relatively small increase in the expected dynamic range (which is estimated be about 25-fold).

Human Study

BLSC and ELSC Isolation And Count: Human blood was obtained by venipuncture following standard acceptable medical practices. Four ml of blood was withdrawn using sterile procedures and placed into tubes containing a 15% EDTA solution. The tubes were inverted three-four times to mix the blood with the EDTA and then placed into refrigeration at 4° C. for 48 hours. After 48 hours of gravity separation, the blood had separated into a floating plasma fraction and a sedimented cellular fraction. The cellular fraction contained red blood cells, white blood cells, most ELSCs, GLSCs, and hematopoietic stem cells. The plasma fraction was withdrawn by a sterile pipette to a second sterile tube and stored under refrigeration at 4° C. Fifteen microliters of the plasma fraction from each human was mixed with 15 microliters of sterile 0.4% Trypan blue, the resultant solution mixed, placed onto a hemocytometer and the isolated cells counted and photographed. Blastomere-like stem cells (BLSCs) are Trypan blue positive and <2.0 microns in size. Blastomere to epiblast transitional cells (BE-TrSCs) are both Trypan blue positive & negative and 3-5 microns in size. Epiblast-like stem cells (ELSCs) are Trypan blue negative and 6-8 microns in size

Initial Observations: The average human BLSC population in circulating blood showed some variability and was in most cases in the range of 300-500×10̂6 cells per ml when drawn from human volunteers with self-assessed status of ‘reasonably healthy’. Remarkably, one volunteer diagnosed with systemic lupus erythematosus had a BLSC count of >800×10̂6 cells ml and did not undergo traditional methotrexate therapy. In contrast, a second volunteer was on methotrexate therapy for a mixed connective tissue disorder and exhibited a significantly lower BLSC count of about 70×10̂6 cells per ml. It should be noted that at this time is remains unclear whether methotrexate therapy impacts the number of BLSCs in peripheral blood.

Contemplated Additional Human Study

Based upon the above and other results, it is contemplated that human patients exhibit similar stem cell mobilization following trauma, and that the number and types of circulating adult stem cells can be correlated with numerous factors, including the severity of trauma, recovery time, choice of treatment options, efficacy of treatment, and prognosis for the patient. Therefore, it should be noted that stem cell mobilization (e.g., quantity, kinetic, duration, etc.) can also be correlated with the expected prognosis, morbidity, and/or mortality of the patient.

General Method: Blood samples collected at specific time periods from trauma patients during an ongoing trauma trial will be used. The samples will be processed for the isolation, identification, and quantification of adult stem cells. In a typical process, 0.5 ml of blood will be hemolyzed and then clarified with Hemolysis Solution and Clarification Solution (MBC-ASB-REBG-900A-001, Moraga Biotech Corp.). It should be noted that the reagents used herein specifically preserve the structure and function of adult stem cells. The supernatants will be discarded and the cell pellets reconstituted. The final cell suspension will be incubated with fluorescently-tagged cluster of differentiation (CD) antibodies to identify cell surface epitopes that permit distinguishing the three types of stem cells. The identity and quantity of germ layer lineage stem cells (i.e., GLSCs), pluripotent stem cells (ELSCs), and totipotent stem cells (BLSCs) will be determined using a flow cytometer. Similar processes will be performed for tissue analysis of stem cells substantially following a protocol as outlined above. The so obtained data will be statistically analyzed to ascertain which type(s) and quantities of adult stem cells were mobilized into the circulatory system following trauma, and optionally what the mobilization kinetics (e.g., absolute and/or relative change in concentration over time). Graphic or other analysis will then assist in correlation with contemplated parameters for various diseases and conditions.

Exemplary Protocol

The following protocol is one example of how a correlation between a stem cell count and a selected disease or condition parameter can be established. Such correlation can then be employed in the assessment of severity of trauma, prognosis, choice of treatment option, and other parameters.

INCLUSION CRITERIA: Patient are classified as a trauma code by an emergency physician and are predicted to be admitted to the hospital for forty-eight hours or longer. Patient or patient's agent must give consent for blood draw.

EXCLUSION CRITERIA: Patients under the age of 18, pregnant patients, patients currently receiving immunosuppressive treatments, and patients not expected to be admitted for more than 48 hours will not be included.

COLLECTION: Once the patient arrives at the emergency center, advanced trauma life support guidelines will commence as per routine. During the initial collection of blood for routine trauma laboratories an additional 10 milliliters of blood will be collected in a separate vial and placed on ice. 10 milliliters of blood will be redrawn at 12, 24, 48, and 72 (if in hospital) hours post trauma and placed on ice. The blood will be stored on ice in said coolers until processed. Optionally, and where appropriate, muscle biopsies are taken at the same time intervals and also stored on ice.

PROCESSING/HARVESTING STEM CELLS: Under sterile conditions, a total of 49.5 mls of Hemolysis solution (MBC-ASB-REBG-900A-001, Moraga Biotech Corp.) is added to a 50 ml conical tube. Using sterile procedure, ½ ml of blood is added to the same conical tube. The tube is inverted twice to mix and hemolyze the red blood cells. The conicals are balanced and spun at 1800×g for 10 minutes. The supernatant is carefully aspirated off of the pellet. The pellet is resuspended. The cells are reconstituted with 2 mls of BLSC Media (MBC-PGB-MED-1A00-A006, Moraga Biotech Corp.) sequentially. The measured final volume determines the number of 50 ml conicals to be used for clarification. 1 ml of cells is added to 49 mls of Clarification solution (MBC-ASB-REBG-900A-001, Moraga Biotech Corp.). The conical tube is inverted twice to mix well. The conicals are balanced and spun at 1800×g for 10 minutes. The supernatant is carefully aspirated off of the pellet. The pellet is resuspended. The cells are reconstituted with 2 mls of serum from the patient's blood. The cells are then counted using microscopy. Alternatively, the reconstituted sample could be equally divided into three aliquots and stained with CD90 (GLSCs), CD10 (ELSCs), and CD66e (BLSCs). Substantially similar procedure as described above is used for muscle biopsy samples, and cells are counted using microscopy as above.

Anticipated Results: It is expected to see increased numbers of the different types of adult stem cells as the severity of trauma increases. Based on previous animal results, it is also expected to see a peak in the total number of stem cells present in the blood within 12 hours after trauma. Adult stem cells are expected to be found in the blood of human trauma patients in higher numbers in patients who exhibit a higher injury severity score. Correlation between the pattern of the stem cell response and the injury severity score will permit the use of the pattern of the stem cell response following trauma as a prognostic indicator for the outcome of treatment, and/or provide a window of treatment with previously prepared/isolated stem cells. Additional data, observations, and further contemplated aspects are disclosed in our copending U.S. patent application with the Ser. No. 11/574,622, and International application with the serial number PCT/US07/05142 (published as WO 07/100845), which form express part of this disclosure.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of medical evaluation, comprising:
a step of ascertaining that a patient has a trauma or disorder, and obtaining at least one of a peripheral stem cell count and a skeletal muscle stem cell count in the patient; and
correlating the stem cell count with at least one or a severity of a disease or trauma, a future treatment option, a prognosis, and/or an anticipated time to recovery.

2. The method of claim 1 wherein the stem cell count is obtained from peripheral blood.

3. The method of claim 1 wherein the stem cell count includes stem cells selected from the group consisting of BLSC and ELSC.

4. The method of claim 3 wherein the stem cell count comprises a step of enrichment of stem cells, and wherein the enrichment substantially exclusively relies on at least one of sedimentation, filtration, and non-optically assisted electrostatic separation.

5. The method of claim 1 wherein the patient is a human, and wherein the disorder is a chronic disease.

6. The method of claim 1 wherein the patient is a human, and wherein the trauma is an acute trauma.

7. The method of claim 1 wherein the patient is a human, and wherein the trauma is surgical intervention.

8. The method of claim 1 further comprising a step of adjusting a treatment protocol in response to the stem cell count.

9. The method of claim 1 further comprising a step of providing to the patient a stem cell preparation in response to the stem cell count.

10. The method of claim 9 wherein the stem cell preparation comprises a BLSC or an ELSC.

11. The method of claim 10 wherein the stem cell preparation is prepared by obtaining whole blood from a patient and allowing the whole blood to sediment for a time sufficient to separate non-stems cells from the stem cells, and by withdrawing the stem cells from a supernatant of the whole blood.

12. The method of claim 1 wherein the patient is a non-human mammal.

13. The method of claim 1 wherein the step of obtaining the stem cell count comprises
obtaining whole blood from a patient and allowing the whole blood to sediment for a time sufficient to separate non-stems cells from the stem cells;
withdrawing the stem cells from a supernatant of the whole blood; and
counting the stem cells to thereby arrive at the stem cell count.

14. The method of claim 11 wherein the whole blood is combined with complexing agent to complex calcium ions.

15. The method of claim 11 wherein the step of allowing the whole blood to sediment is performed at a temperature below room temperature for at least 48 hours.

16. The method of claim 11 wherein the time sufficient to separate is sufficient to provide a stem cell population in the supernatant that has less than 1% non-stem cells.

17. The method of claim 1 wherein the stem cell is a BLSC or an ELSC.
